(19) United States
(12) Patent Application Publication
(10) Pub. No.: US 2021/0032307 A1
(43) Pub. Date: Feb. 4, 2021

(54) CODON OPTIMIZED PRECURSOR GENE AND SIGNAL PEPTIDE GENE OF HUMAN INSULIN ANALOGUE

(71) Applicant: JIANGSU HENGRUI MEDICINE CO., LTD., Lianyungang (CN)

(72) Inventors: Feifei WANG, Lianyungang (CN); Lei CHEN, Lianyungang (CN); Hongwei WANG, Lianyungang (CN)

(21) Appl. No.: 16/967,117

(22) PCT Filed: Feb. 1, 2019
(86) PCT No.: PCT/CN2019/074384

§ 371 Date: Aug. 3, 2020

(30) Foreign Application Priority Data
Feb. 9, 2018 (CN) 201810131794.3

(51) Int. Cl. C07K 14/62; C12N 15/81

(52) U.S. Cl. CPC C07K 14/62; C12N 15/815

(57) ABSTRACT

Provided is a nucleic acid molecule of a codon optimized precursor gene and signal peptide gene of a human insulin analogue. The nucleic acid molecule comprises a nucleic acid molecule encoding the precursor of the fusion insulin analogue and a nucleic acid molecule encoding the yeast secreting signal peptide α-factor. The nucleic acid molecule improves the expression of the precursor of the insulin analogue in Pichia Pastoris, and reduces the production cost of the human insulin analogue.

### FIELD OF THE INVENTION

The present invention relates to a codon-optimized human insulin analogue precursor gene and a codon-optimized α-factor signal peptide gene, and provides a method for expressing the human insulin analogue precursor gene.

### BACKGROUND OF THE INVENTION

Human insulin is a polypeptide consisting of 51 amino acids and comprises two chains, Chain A and Chain B, respectively. The main efficiency of insulin is to regulate glucose metabolism. Insulin intervention is the most direct and effective method as an alternative or supplementary treatment for diabetes. Insulin also has effect on promoting synthesis of fat, inhibiting decomposition of fat, and reducing the production of ketone body, thereby it is also used to correct various symptoms of insulin-related ketosis and acidosis.

Insulin was previously extracted from the pancreas of pigs, bovine and other animals, but these products are structurally different from human insulin, so they have immunogenicity. Since genetically recombinant technology for production of human insulin was developed by Eli Lilly & Co. USA and Novo Nordisk Denmark successively, in the early and mid-1980s, genetically expressing human insulin and its analogues became the main means in the industry. However, it is extremely inconvenient for patients who need to be injected frequently with human insulin due to the short-term effect of insulin. Therefore, efforts have been made to obtain insulin analogues and derivatives with long-term effect in human. Among them, modification of human insulin or analogue thereof with acylated group is an effective method for increasing the half-life thereof. A human insulin analogue was disclosed in WO2018024186, in which position B29 was substituted with a long chain fatty acid and the amino acid at position B30 was deleted, and the structure and biological activity of the human insulin analogue were disclosed. An insulin analogue having a 14-acyl side chain linked to position B29 and an amino acid deletion at position B30, and a preparation thereof were disclosed in WO9507931. A human insulin analogue in which B29 position was substituted with a glutamic acid and a long chain fatty acid, and the amino acid at position B30 was deleted, was disclosed in WO2005012347. At present, the most commonly used expression systems for expressing human insulin and analogue thereof are Escherichia coli, Saccharomyces Cerevisiae and Pichia Pastoris, among which, human insulin and analogue thereof is expressed in the form of inclusion body in E. coli, and cleavage and renaturation of inclusion body are necessary, which makes the process cumbersome and low-yield; S. cerevisiae and P. pastoris have the advantages due to the ease of operation, ease of cultivation, modification of foreign protein, and the capability of secretion expression, and so forth. However, the secretion efficiency for Saccharomyces Cerevisiae is low and the strain for expression is not stable. By contrast, Pichia Pastoris is an expression system more widely used in industrial production of recombinant proteins. Industrially, the fermentation yield during the production process is a key factor in controlling the production cost. Due to the large demand in commercial insulin, in Novo Nordisk, a major producer, the scale of the tank reaches dozens of tons for production in yeast expression system, which involves high requirements for both plant and equipment and finally results in high cost. Thus, it is of great significance to increase the fermentation yield of human insulin and analogue thereof in industrial production.

Genetic codon is a triplet code consisting of three adjacent bases on the messenger ribonucleic acid (mRNA). There are 64 types of genetic codons. However, frequency of codon usage differs for different organisms, even for different protein-coding genes of the same organism, i.e., there is codon preference. Codons of foreign genes mainly affect gene expression at the translational level. There are many literatures showing that codon optimization has significant effect on increasing the expression of foreign proteins in Pichia Pastoris. Exogenous genes are expressed in Pichia Pastoris in the form of intracellular expression and secretory expression. For the latter, signal peptides are required to direct the secretion of products expressed by exogenous genes. Currently, the most commonly used signal peptide is derived from α-factor signal peptide of Saccharomyces Cerevisiae, and the nucleotide sequence thereof is also derived from Saccharomyces Cerevisiae. α-factor signal peptide nucleotide sequence optimized for Pichia Pastoris has not been reported so far. There are many related literatures on codon optimization of insulin precursors, for example, an optimized human insulin precursor gene sequence and its expression in Pichia Pastoris by Gurramkonda et al. (Gurramkonda et al. Application of simple fed-batch technique to high-level secretory production of insulin precursor using Pichia pastoris with subsequent purification and conversion to human insulin. Microbial Cell Factories, 2010, 9:31), and patent publication WO1998028429 discloses a gene sequence expressing a human insulin analogue precursor, and the insulin precursor amino acid sequence encoded by the gene is EEGEPK-B(1-29)-AAK-A(1-21), wherein EEGEPK is an N-terminal extension of the insulin precursor, referred to as spacer peptide or leader peptide, which is capable of protecting the N-terminus of the insulin precursor from the hydrolysis via yeast protease, and is capable of improving the expression efficiency of the insulin precursor; B (1-29) is human insulin Chain B with deletion of B30 threonine; A (1-21) is the amino acid sequence of human insulin Chain A; AAK is a linker peptide linking Chain B to Chain A, also referred to as C peptide.

In order to further increase the yield of human insulin and its analogue precursors, the inventors optimized the insulin analogue precursor gene and the α-factor signal peptide gene for secretory expression in Pichia Pastoris according to the codon preference in Pichia Pastoris. Our results show that the yield of the human insulin analogue precursor was increased by almost two fold by the codon-optimized gene expression according to the present invention, when compared with the human insulin analogue precursor gene known in the prior art (as a control). The cost of industrial production of human insulin and its analogues will be greatly reduced in the late stage.

### SUMMARY OF THE INVENTION

In some embodiments of the invention, provides a nucleic acid molecule comprising the following structure:

5′-(PS)ₐ-(SP)_{b}-(LS)_{c}-GE-(P′S)_{d}-3′,

wherein PS is a nucleic acid molecule encoding a processing site, a is 0 or 1;

SP is a nucleic acid molecule encoding signal peptide, b is 0 or 1;

LS is a nucleic acid molecule encoding spacer peptide, c is 0 or 1;

GE is a nucleic acid molecule encoding polypeptide of interest; and

P′S is a nucleic acid molecule encoding processing site, and d is 0 or 1.

In some embodiments, provides a nucleic acid molecule comprising the following structure:

5′-(PS)ₐ-(SP)_{b}-(LS)_{c}-GE-(P′S)_{d}-3′,

wherein PS is a nucleic acid molecule encoding processing site, a is 0 or 1;

SP is a nucleic acid molecule encoding signal peptide, b is 1;

LS is a nucleic acid molecule encoding spacer peptide, c is 1;

GE is a nucleic acid molecule encoding polypeptide of interest; and

P′S is a nucleic acid molecule encoding processing site, and d is 0 or 1.

In some embodiments, the nucleic acid molecule encoding signal peptide comprises the sequence shown as SEQ ID NO: 1.

In some embodiments, the polypeptide of interest is a human insulin analogue precursor polypeptide; the nucleic acid molecule encoding the human insulin analogue precursor polypeptide comprises the sequence shown as SEQ ID NO: 3.

In some embodiments, the nucleic acid sequence of the nucleic acid molecule encoding signal peptide (SP) is shown as SEQ ID NO: 1, and the amino acid sequence thereof is shown as SEQ ID NO: 2:

SEQ ID NO: 1
ATGAGATTTCCTTCTATTTTCACTGCTGTTTTGTTTGCCGCTTCCTCTGC
TTTGGCAGCTCCAGTTAATACAACCACTGAAGATGAGACTGCTCAAATCC
CAGCCGAAGCAGTTATTGGTTACTCCGACTTGGAAGGAGATTTTGACGTC
GCTGTTTTACCATTCTCTAATTCCACTAATAACGGTCTGTTGTTTATTAA
TACTACCATTGCTTCTATCGCCGCTAAGGAGGAAGGTGTGTCCCTCGAGA
AAAGA;
SEQ ID NO: 2
MRFPSIFTAVLFAASSALAAPVNTTTEDETAQIPAEAVIGYSDLEGDFDV
AVLPFSNSTNNGLLFINTTIASIAAKEEGVSLEKR;

In some embodiments, the nucleic acid molecule encoding polypeptide of interest (GE) may be a nucleic acid molecule encoding human insulin analogue precursor, wherein the human insulin analogue precursor may be human insulin with a deletion of threonine at position B30. The nucleic acid molecule sequence of the human insulin analogue precursor is shown as SEQ ID NO:3, and the amino acid sequence thereof is shown as SEQ ID NO: 4:

SEQ ID NO: 3
TTCGTCAACCAGCACTTGTGTGGTTCCCATTTGGTTGAGGCTCTGTACTT
GGTCTGTGGAGAAAGAGGTTTCTTTTACACCCCAAAGGCTGCTAAGGGTA
TCGTTGAGCAATGTTGCACCTCTATTTGTTCCCTGTATCAGTTGGAAAAC
TACTGCAACTAA;
SEQ ID NO: 4
FVNQHLCGSHLVEALYLVCGERGFFYTPKAAKGIVEQCCTSICSLYQLEN
YCN.

In some embodiments, positions 88-96 of the nucleic acid molecule encoding human insulin analogue precursor (i.e., GCTGCTAAG) presents a nucleic acid molecule encoding linker peptide (also referred to as C-peptide), which may be substituted with the following sequence, including but not limited to: GCCGCTAAG, GCTGCCAAG, GCTGCTAAA, GCCGCCAAG.

In some embodiments, the sequence of the nucleic acid molecule (LS) encoding spacer peptide is shown as SEQ ID NO: 5.

SEQ ID NO: 5
gaagaaggtgaaccaaag.

In some embodiments, the PS and/or P′S are nucleic acid molecules encoding restriction site.

Preferably, PS is a nucleic acid molecule encoding EcoR I restriction site, and/or P′S is a nucleic acid molecule encoding Not I restriction site.

In some embodiments, provides a nucleic acid molecule capable of expressing human insulin analogue precursor, wherein the nucleic acid molecule comprises a nucleic acid molecule encoding spacer peptide and a nucleic acid molecule encoding human insulin analogue precursor, and is capable of expressing human insulin analogue precursor, after recombination with a vector comprising a signal peptide.

In some embodiments, the amino acid sequence of the human insulin analogue precursor encoded by the human insulin analogue precursor nucleic acid molecule is as follows:

EEGEPK-B(1-29)-AAK-A(1-21)

wherein “EEGEPK (SEQ ID NO: 16)” may be a N-terminal extension of the insulin precursor, referred to as spacer peptide or leader peptide; “B(1-29)” may be a human insulin Chain B with a deletion of threonine on position B30. “A(1-21)” may be a human insulin Chain A amino acid sequence, and “AAK” is a linker peptide linking Chain B to Chain A, also referred to as C peptide.

In some embodiments, the sequence of the human insulin analogue precursor nucleic acid molecule may be shown as SEQ ID NO: 6, and the amino acid sequence thereof is shown as SEQ ID NO: 7:

SEQ ID NO: 6
GAAGAAGGTGAACCAAAGTTCGTCAACCAGCACTTGTGTGGTTCCCATT
TGGTTGAGGCTCTGTACTTGGTCTGTGGAGAAAGAGGTTTCTTTTACAC
CCCAAAGGCTGCTAAGGGTATCGTTGAGCAATGTTGCACCTCTATTTGT
TCCCTGTATCAGTTGGAAAACTACTGCAACTAA;
SEQ ID NO: 7
EEGEPKFVNQHLCGSHLVEALYLVCGERGFFYTPKAAKGIVEQCCTSIC
SLYQLENYCN.

In some embodiments, another nucleic acid molecule capable of expressing human insulin analogue precursor is provided. The nucleic acid molecule sequence comprises a signal peptide sequence, a spacer peptide sequence, and a sequence encoding human insulin analogue precursor, and the nucleic acid molecule is capable of expressing human insulin analogue precursor after recombination with a vector comprising no signal peptide.

In some embodiments, the nucleic acid sequence of the nucleic acid molecule expressing human insulin analogue precursor is shown as SEQ ID NO: 8, and the encoded amino acid sequence is shown as SEQ ID NO:9:

SEQ ID NO: 8
ATGAGATTTCCTTCTATTTTCACTGCTGTTTTGTTTGCCGCTTCCTCTG
CTTTGGCAGCTCCAGTTAATACAACCACTGAAGATGAGACTGCTCAAAT
CCCAGCCGAAGCAGTTATTGGTTACTCCGACTTGGAAGGAGATTTTGAC
GTCGCTGTTTTACCATTCTCTAATTCCACTAATAACGGTCTGTTGTTTA
TTAATACTACCATTGCTTCTATCGCCGCTAAGGAGGAAGGTGTGTCCCT
CGAGAAAAGAGAAGAAGGTGAACCAAAGTTCGTCAACCAGCACTTGTGT
GGTTCCCATTTGGTTGAGGCTCTGTACTTGGTCTGTGGAGAAAGAGGTT
TCTTTTACACCCCAAAGGCTGCTAAGGGTATCGTTGAGCAATGTTGCAC
CTCTATTTGTTCCCTGTATCAGTTGGAAAACTACTGCAACTAA;
SEQ ID NO: 9
MRFPSIFTAVLFAASSALAAPVNTTTEDETAQIPAEAVIGYSDLEGDFD
VAVLPFSNSTNNGLLFINTTIASIAAKEEGVSLEKREEGEPKFVNQHLC
GSHLVEALYLVCGERGFFYTPKAAKGIVEQCCTSICSLYQLENYCN.

In some embodiments, the nucleic acid molecule expressing human insulin analogue precursor may also comprise restriction site(s), preferably the restriction site(s) is (are) EcoR I restriction site and/or Not I restriction site.

In some embodiments, a vector capable of being expressed in a eukaryotic or prokaryotic cell is provided, which is capable of expressing human insulin analogue precursor in a prokaryotic or eukaryotic cell via secretory expression.

In some embodiments, a host cell is also provided. Preferably, the host cell is yeast, more preferably Pichia, which is capable of expressing human insulin analogue precursor via secretory expression.

In some embodiments, a method for preparing a human insulin analogue is also provided, comprising utilizing the nucleic acid molecule, vector, and/or host cell as described above.

The method may further comprise the following steps:

1) expressing a human insulin analogue precursor in a eukaryotic cell by a nucleic acid molecule encoding the human insulin analogue precursor;

2) obtaining a human insulin analogue by enzymatically digesting the human insulin analogue precursor, the nucleic acid molecule encoding the human insulin analogue precursor may be shown as SEQ ID NO: 6, and the insulin analogue precursor may be enzymatically digested by using the methods well known to those skilled in the art.

In some embodiments, step 1) comprises expressing the human insulin analogue precursor by an expression vector comprising a signal peptide sequence, and said signal peptide sequence is shown as SEQ ID NO: 1.

In some embodiments, the human insulin analogue is a human insulin with deletion of B30, and the human insulin analogue is further substituted with an acylated group.

In some embodiments, within said human insulin with deletion of B30, the lysine at position B29 is substituted by the acylated group.

Preferably, the product obtained after the above substitution is lysine B29 (N^{ε}-(N^{α}-hexadecane fatty diacid-L-lysine-N^{ε}-oxobutylyl)) des(B30) human insulin.

### Abbreviations and Terms

“Codon optimization” refers to the synthesis of genes with preferred codons instead of codons with low frequency or rare codons according to the rule of codon preference for host cells.

“Control 1” is shown as SEQ ID NO: 10 below, wherein a nucleic acid molecule encoding “EEGEPK” (GAAGAAGGTGAACCAAAG, double underlined) is linked to a nucleic acid molecule encoding the insulin precursor gene taught by WO1998028429:

SEQ ID NO: 10
GAAGAAGGTGAACCAAAGTTCGTTAACCAACACTTGTGCGGTTCCCACT
TGGTTGAAGCTTTGTACTTGGTTTGCGGTGAAAGAGGTTTCTTCTACAC
TCCTAAGGCTGCTAAGGGTATTGTCGAACAATGCTGTACCTCCATCTGC
TCCTTGTACCAATTGGAAAACTACTGCAACTAA.

“Control 2” is shown as SEQ ID NO: 11 below, wherein a nucleic acid molecule encoding “EEGEPK” (double underlined) is linked to a nucleic acid molecule encoding the optimized insulin precursor gene taught by Gurramkonda et al. (Microbial Cell Factories, 2010, 9:31):

SEQ ID NO: 11
GAAGAAGGTGAACCAAAGTTTGTTAACCAACATTTGTGTGGTTCTCATT
TGGTTGAAGCTTTGTACTTGGTTTGTGGTGAAAGAGGTTTCTTCTACAC
TCCAAAGGCTGCTAAGGGTATTGTTGAACAATGTTGTACTTCTATTTGT
TCTTTGTACCAATTGGAAAACTACTGTAACTAA.

“IP-S” is a nucleic acid molecule corresponding to an insulin precursor gene after codon optimization.

“α-factor” is a nucleic acid molecule corresponding to the α-factor signal peptide gene comprised in pPIC9K Expression Vector provided by Invitrogen, which is derived from Saccharomyces Cerevisiae.

“α-factor-S” is a nucleic acid molecule corresponding to a codon-optimized α-factor signal peptide gene.

“Vector” includes nucleic acid molecule, which is capable of transporting another nucleic acid to which it is linked, including but not limited to, plasmid and viral vector. Some vectors are capable of autonomously replicating in the host cell into which they are introduced, while others can be integrated into the genome of the host cell upon introduction into the host cell and thereby being replicated along with the host genome. In addition, some vectors are capable of directing the expression of genes operably linked thereto, and such vectors are referred to herein as “recombinant expression vectors” (or simply as “expression vectors”). Traditional vectors are well known in the art.

“Polypeptide of interest” is a polypeptide that can be expressed in yeast, including but not limited to enzyme, antibody, interferon, insulin, interleukin, and the like, and variant, precursor, intermediate thereof. For example, polypeptide of interest may be insulin precursor.

“Cell” and “host cell” may be interchangeably used.

“Polynucleotide molecule”, “nucleic acid molecule” may be interchangeably used and the sequence thereof may be DNA sequence.

### DETAILED DESCRIPTION OF THE DISCLOSURE

The following examples are provided to further illustrate the invention, but are not intended to limit the scope of the invention.

The vectors, host bacteria and culture media used in the examples of the present invention were purchased from Invitrogen. pPIC9K, a Pichia Pastoris expression vector, contains an alcohol oxidase AOX1 promoter, which can be induced by methanol, and the vector also contains α-factor signal peptide sequence and is capable of expressing foreign proteins via secretory expression. pPIC3.5K, another Pichia Pastoris expression vector, contains alcohol oxidase AOX1 promoter, which can be induced by methanol, and the vector does not contain α-factor signal peptide sequence. Pichia Pastoris GS115 strain was used as host bacteria. The formulation of the culture medium was provided by the Pichia Pastoris manual.

### EXAMPLE 1

### Construction of Recombinant Expression Vector for Insulin Precursor

EcoR I and Not I restriction sites were added into the 5′- and 3′-ends of Control 1 (SEQ ID NO: 10), Control 2 (SEQ ID NO: 11) and IP-S (SEQ ID NO: 6), respectively and synthesized by Nanjing Kingsray Biotech Co., Ltd. The synthesized nucleic acid molecule sequence was ligated into T vector.

SEQ ID NO: 6
GAAGAAGGTGAACCAAAGTTCGTCAACCAGCACTTGTGTGGTTCCCATT
TGGTTGAGGCTCTGTACTTGGTCTGTGGAGAAAGAGGTTTCTTTTACAC
CCCAAAGGCTGCTAAGGGTATCGTTGAGCAATGTTGCACCTCTATTTGT
TCCCTGTATCAGTTGGAAAACTACTGCAACTAA;
SEQ ID NO: 10
GAAGAAGGTGAACCAAAGTTCGTTAACCAACACTTGTGCGGTTCCCACT
TGGTTGAAGCTTTGTACTTGGTTTGCGGTGAAAGAGGTTTCTTCTACAC
TCCTAAGGCTGCTAAGGGTATTGTCGAACAATGCTGTACCTCCATCTGC
TCCTTGTACCAATTGGAAAACTACTGCAACTAA;
SEQ ID NO: 11
GAAGAAGGTGAACCAAAGTTTGTTAACCAACATTTGTGTGGTTCTCATT
TGGTTGAAGCTTTGTACTTGGTTTGTGGTGAAAGAGGTTTCTTCTACAC
TCCAAAGGCTGCTAAGGGTATTGTTGAACAATGTTGTACTTCTATTTGT
TCTTTGTACCAATTGGAAAACTACTGTAACTAA.

The T vector carrying the insulin precursor nucleic acid molecule and the expression vector pPIC9K were digested with both EcoR I and Not I, and then the target fragment and the vector fragment were separately recovered by a Gel Recovery Kit. After purification of the digested fragments, and the target fragment was ligated to the vector pPIC9K with T4 ligase.

The above-mentioned ligation solution was transformed into E. coli TOP10 competent cells, and plated onto a plate with ampicillin resistance. After cultivation, the bacterial colony was picked, and the plasmid was extracted and verified by digestion with both restriction enzymes. Three recombinant expression vectors comprising Control 1, Control 2 and IP-S sequence respectively, were finally obtained.

### EXAMPLE 2

### Expression of Insulin Precursor by Pichia Pastoris Recombinant Strain

The three recombinant expression vectors constructed in Example 1 were transformed into Pichia Pastoris GS115 respectively, the recombinant strains expressing Control 1 and Control 2 were used as control strains, and the recombinant strain expressing IP-S was used as test strain.

The colonies from the three recombinant bacteria were inoculated into 5 mL YPD medium, and cultivated at a constant temperature of 30° C. while shaking at 250 rpm, until the value of OD₆₀₀ reached about 10 (16-18 hours). The cells were collected and resuspended in 50 mL BMGY medium, and cultivated overnight at a constant temperature of 30° C. while shaking at 250 rpm, until the value of OD₆₀₀ reached about 30. The cells were collected by centrifuging at 1500 rpm for 5 minutes and resuspended in 25 mL of BMMY medium. 1/200 volume of methanol (final concentration of 0.5%) was added into the medium, and cultivated at a constant temperature of 30° C. while shaking at 250 rpm for 96 hours, while 1/200 volume of methanol was supplemented every 24 hours. Upon the expression was finished, the supernatant was obtained after centrifugation at 10,000 rpm. The yield of the insulin precursor comprised in the supernatant was measured by HPLC, and converted into a percentage relative to the expression amount of the insulin precursor expressed by the control strain. The percentage of expression level of the insulin precursor is shown as Table 1.

TABLE 1

| | | Signal | Expression | Percentage of |
|---|---|---|---|---|
| Bacterial strain | vector | peptide | Gene | yield (%) |
| Control bacterium | pPIC9K | α-factor | Control 1 | 100 |
| Control bacterium | pPIC9K | α- factor | Control 2 | 125 |
| Test bacterium | pPIC9K | α- factor | IP-S | 225 |

The data in Table 1 shows that the amount of insulin precursor expressed by the optimized insulin precursor gene was increased by 1.8 to 2.25 times compared to those in the two control groups. It can be seen that the optimized insulin precursor gene has superior expression efficiency and can significantly improve the yield of the expressed insulin precursor.

### EXAMPLE 3

### Construction of Recombinant Expression Vectors Comprising Insulin Precursor Gene Fused to Different α-Factors

Nucleic acid molecules shown as SEQ ID NO: 13 and SEQ ID NO: 8, in which α-factor (SEQ ID NO: 12) and α-factor-S (SEQ ID NO: 1) were separately fused to the site in the upstream of IP-S, were synthesized. EcoR I and Not I restriction sites were also incorporated at both 5′ and 3′ ends of the synthesized nucleic acid molecules. The synthesized nucleic acid molecules were ligated to the T vector.

SEQ ID NO: 12
ATGAGATTTCCTTCAATTTTTACTGCAGTTTTATTCGCAGCATCCTCCG
CATTAGCTGCTCCAGTCAACACTACAACAGAAGATGAAACGGCACAAAT
TCCGGCTGAAGCTGTCATCGGTTACTCAGATTTAGAAGGGGATTTCGAT
GTTGCTGTTTTGCCATTTTCCAACAGCACAAATAACGGGTTATTGTTTA
TAAATACTACTATTGCCAGCATTGCTGCTAAAGAAGAAGGGGTATCTCT
CGAGAAAAGA
SEQ ID NO: 1
ATGAGATTTCCTTCTATTTTCACTGCTGTTTTGTTTGCCGCTTCCTCTG
CTTTGGCAGCTCCAGTTAATACAACCACTGAAGATGAGACTGCTCAAAT
CCCAGCCGAAGCAGTTATTGGTTACTCCGACTTGGAAGGAGATTTTGAC
GTCGCTGTTTTACCATTCTCTAATTCCACTAATAACGGTCTGTTGTTTA
TTAATACTACCATTGCTTCTATCGCCGCTAAGGAGGAAGGTGTGTCCCT
CGAGAAAAGA
SEQ ID NO: 13
ATGAGATTTCCTTCAATTTTTACTGCAGTTTTATTCGCAGCATCCTCCG
CATTAGCTGCTCCAGTCAACACTACAACAGAAGATGAAACGGCACAAAT
TCCGGCTGAAGCTGTCATCGGTTACTCAGATTTAGAAGGGGATTTCGAT
GTTGCTGTTTTGCCATTTTCCAACAGCACAAATAACGGGTTATTGTTTA
TAAATACTACTATTGCCAGCATTGCTGCTAAAGAAGAAGGGGTATCTCT
CGAGAAAAGAGAAGAAGGTGAACCAAAGTTCGTCAACCAGCACTTGTGT
GGTTCCCATTTGGTTGAGGCTCTGTACTTGGTCTGTGGAGAAAGAGGTT
TCTTTTACACCCCAAAGGCTGCTAAGGGTATCGTTGAGCAATGTTGCAC
CTCTATTTGTTCCCTGTATCAGTTGGAAAACTACTGCAACTAA
SEQ ID NO: 8
ATGAGATTTCCTTCTATTTTCACTGCTGTTTTGTTTGCCGCTTCCTCTG
CTTTGGCAGCTCCAGTTAATACAACCACTGAAGATGAGACTGCTCAAAT
CCCAGCCGAAGCAGTTATTGGTTACTCCGACTTGGAAGGAGATTTTGAC
GTCGCTGTTTTACCATTCTCTAATTCCACTAATAACGGTCTGTTGTTTA
TTAATACTACCATTGCTTCTATCGCCGCTAAGGAGGAAGGTGTGTCCCT
CGAGAAAAGAGAAGAAGGTGAACCAAAGTTCGTCAACCAGCACTTGTGT
GGTTCCCATTTGGTTGAGGCTCTGTACTTGGTCTGTGGAGAAAGAGGTT
TCTTTTACACCCCAAAGGCTGCTAAGGGTATCGTTGAGCAATGTTGCAC
CTCTATTTGTTCCCTGTATCAGTTGGAAAACTACTGCAACTAA

Nucleic acid molecules shown as SEQ ID NO: 14 and SEQ ID NO: 15, in which α-factor (SEQ ID NO: 12) and α-factor-S (SEQ ID NO: 1) were separately fused to the site in the upstream of control 1, were synthesized. EcoR I and Not I restriction sites were also incorporated at both 5′ and 3′ ends of the synthesized nucleic acid molecules. The synthesized nucleic acid molecules were ligated to the T vector.

SEQ ID NO: 14
ATGAGATTTCCTTCAATTTTTACTGCAGTTTTATTCGCAGCATCCTCCG
CATTAGCTGCTCCAGTCAACACTACAACAGAAGATGAAACGGCACAAAT
TCCGGCTGAAGCTGTCATCGGTTACTCAGATTTAGAAGGGGATTTCGAT
GTTGCTGTTTTGCCATTTTCCAACAGCACAAATAACGGGTTATTGTTTA
TAAATACTACTATTGCCAGCATTGCTGCTAAAGAAGAAGGGGTATCTCT
CGAGAAAAGAGAAGAAGGTGAACCAAAGTTCGTTAACCAACACTTGTGC
GGTTCCCACTTGGTTGAAGCTTTGTACTTGGTTTGCGGTGAAAGAGGTT
TCTTCTACACTCCTAAGGCTGCTAAGGGTATTGTCGAACAATGCTGTAC
CTCCATCTGCTCCTTGTACCAATTGGAAAACTACTGCAACTAA
SEQ ID NO: 15
ATGAGATTTCCTTCTATTTTCACTGCTGTTTTGTTTGCCGCTTCCTCTG
CTTTGGCAGCTCCAGTTAATACAACCACTGAAGATGAGACTGCTCAAAT
CCCAGCCGAAGCAGTTATTGGTTACTCCGACTTGGAAGGAGATTTTGAC
GTCGCTGTTTTACCATTCTCTAATTCCACTAATAACGGTCTGTTGTTTA
TTAATACTACCATTGCTTCTATCGCCGCTAAGGAGGAAGGTGTGTCCCT
CGAGAAAAGAGAAGAAGGTGAACCAAAGTTCGTTAACCAACACTTGTGC
GGTTCCCACTTGGTTGAAGCTTTGTACTTGGTTTGCGGTGAAAGAGGTT
TCTTCTACACTCCTAAGGCTGCTAAGGGTATTGTCGAACAATGCTGTAC
CTCCATCTGCTCCTTGTACCAATTGGAAAACTACTGCAACTAA.

The above T vector and the expression vector pPIC3.5K were digested with both endonucleases EcoR I and Not I, and then the target fragment and the vector fragment were separately recovered by Gel Recovery Kit. After purification of the digested fragments, and the target fragment was ligated to the vector pPIC3.5K with T4 ligase.

The above-mentioned ligation solution was transformed into E. coli TOP10 competent cells, and plated onto a plate with ampicillin resistance. After cultivation, the bacterial colony was picked, and the plasmid was extracted and verified by digestion with both restriction enzymes. Four recombinant expression vectors which separately incorporate α-factor or α-factor-S signal peptide for expressing insulin precursor gene with different nucleotide sequences, were finally obtained.

### EXAMPLE 4

### Expression of Insulin Precursor by Pichia Pastoris Recombinant Strain, Before and After Optimization

The recombinant expression vectors constructed in Example 3 were separately transformed into Pichia Pastoris GS115.

The recombinant bacterium colonies were inoculated into 5 mL YPD medium, and cultivated at a constant temperature of 30° C. while shaking at 250 rpm, until the value of OD₆₀₀ reached about 10 (16-18 hours). The cells were collected and re-suspended in 50 mL BMGY medium, and cultivated overnight at a constant temperature of 30° C. while shaking at 250 rpm, until the value of OD₆₀₀ reached about 30. The cells were collected by centrifuging at 1500 rpm for 5 minutes and re-suspended in 25 mL of BMMY medium. 1/200 volume of methanol (final concentration of 0.5%) was added into the medium, and cultivated at a constant temperature of 30° C. while shaking at 250 rpm for 96 hours, while 1/200 volume of methanol was supplemented every 24 hours. Upon the expression was finished, the supernatant was obtained after centrifugation at 10,000 rpm. The yield of the insulin precursor comprised in the supernatant was measured by HPLC.

The recombinant bacterium expressing control 1 gene fused to α-factor was used as a control bacterium. The yield of insulin precursor by other strains was converted into a percentage relative to the yield of the insulin precursor expressed by the control strain, as shown as Table 2.

TABLE 2

| Bacterial | | Signal | Expression | Percentage |
|---|---|---|---|---|
| strain | vector | peptide | Gene | of yield (%) |
| Control | pPIC3.5K | α-factor | Control 1 | 100 |
| bacterium | | | | |
| test bacterium | PPIC3.5K | α-factor -S | Control 1 | 150 |
| test bacterium | PPIC3.5K | α-factor | IP-S | 225 |
| test bacterium | PPIC3.5K | α-factor -S | IP-S | 275 |

The data in Table 2 shows that the yield of the insulin precursor was increased by 1.5 times after merely optimizing the signal peptide in the nucleic acid molecule, and such yield was increased by 2.25 times after merely optimizing the insulin precursor gene. By contrast, the yield of the insulin precursor was increased by 2.75 times after optimizing both the signal peptide and the insulin precursor gene. Taken together, codon optimization can increase the expression level of insulin precursor to 1.5-2.75 times.

1. A nucleic acid molecule comprising molecule or structure having the following general formula:
5′-(PS)ₐ-(SP)_{b}-(LS)_{c}-GE-(P′S)_{d}-3′,
wherein PS is a nucleic acid molecule encoding a processing site, a is 0 or 1;
SP is a nucleic acid molecule encoding a signal peptide, b is 0 or 1;
LS is a nucleic acid molecule encoding a spacer peptide, c is 0 or 1;
GE is a nucleic acid molecule encoding a polypeptide of interest; and
P′S is a nucleic acid molecule encoding a processing site, d is 0 or 1; and
the nucleic acid molecule encoding the signal peptide comprises a sequence shown as SEQ ID NO:1.

2. A nucleic acid molecule comprising molecule or structure having the following general formula:
5′-(PS)ₐ-(SP)_{b}-(LS)_{c}-GE-(P′S)_{d}-3′,
wherein PS is a nucleic acid molecule encoding a processing site, a is 0 or 1;
SP is a nucleic acid molecule encoding a signal peptide, b is 1;
LS is a nucleic acid molecule encoding a spacer peptide, c is 0 or 1;
GE is a nucleic acid molecule encoding a human insulin analogue precursor polypeptide; and
P′S is a nucleic acid molecule encoding a processing site, d is 0 or 1; and
the nucleic acid molecule encoding the human insulin analogue precursor polypeptide comprises sequence shown as SEQ ID NO:3.

3. The nucleic acid molecule according to claim 1, wherein the polypeptide of interest is a human insulin analogue precursor comprising a nucleic acid molecule encoding an amino acid sequence shown as SEQ ID NO: 4.

4. The nucleic acid molecule according to claim 2, wherein the nucleic acid molecule encoding the signal peptide comprises a nucleic acid molecule encoding an amino acid sequence shown as SEQ ID NO: 2.

5. The nucleic acid molecule according to claim 2, wherein the nucleic acid molecule encoding human insulin analogue precursor comprises a substitution at positions 88-96 of SEQ ID NO: 3.

6. The nucleic acid molecule according to claim 1, wherein the amino acid sequence of the spacer peptide comprises SEQ ID NO: 16.

7. The nucleic acid molecule according to claim 1, wherein the nucleic acid molecule:
a) comprises a sequence selected from the group consisting of SEQ ID NO: 1, SEQ ID NO: 3, SEQ ID NO: 6, SEQ ID NO: 8, SEQ ID NO: 13 and SEQ ID NO:15; or
b) consists of a sequence selected from the group consisting of SEQ ID NO: 1, SEQ ID NO: 3, SEQ ID NO: 6, SEQ ID NO: 8, SEQ ID NO: 13 and SEQ ID NO:15.

8. The nucleic acid molecule according to claim 1, wherein the processing site is a restriction site.

9. A vector comprising the nucleic acid molecule according to claim 1.

10. A host cell comprising the vector of claim 9.

11. A method for producing a human insulin analogue comprising the step of cultivating the host cell of claim 10 in a medium.

12. The method of claim 11, further comprising the step of:
1) expressing a human insulin analogue precursor; and
2) obtaining a human insulin analogue by enzymatically digesting the human insulin analogue precursor obtained in step 1).

13. The method according to claim 11, wherein the human insulin analogue is a human insulin with deletion of B30, and/or the human insulin analogue is further substituted with an acylated group.

14. The nucleic acid molecule according to claim 3, wherein the nucleic acid sequence comprises a sequence shown as SEQ ID NO: 3.

15. The nucleic acid molecule according to claim 4, wherein the nucleic acid sequence comprises a sequence shown as SEQ ID NO: 1 or SEQ ID NO: 12.

16. The nucleic acid molecule according to claim 5, wherein the substitution at positions 88-96 of SEQ ID NO: 3 is with GCCGCTAAG, GCTGCCAAG, GCTGCTAAA or GCCGCCAAG.

17. The nucleic acid molecule according to claim 6, wherein the nucleic acid molecule encoding the spacer peptide comprises a sequence shown as SEQ ID NO: 5.

18. The host cell according to claim 10, wherein the host cell is a yeast.

19. The host cell according to claim 18, wherein the yeast is Pichia Pastoris.

20. The method according to claim 13, wherein the lysine at position B29 in the human insulin analogue is substituted with an acylated group.

21. The method according to claim 20, wherein the human insulin analogue after substitution is lysine B29 (N^{ε}-(N^{α}-hexadecane fatty diacid-L-lysine-N^{ε}-oxobutylyl)) des(B30) human insulin.


SEQUENCE LISTING (16 sequences)

SEQ ID NO: 1 - DNA, 255 bp, Artificial Sequence
  GENE nucleic acid molecule encoding signal peptide
atgagatttccttctattttcactgctgttttgtttgccgcttcctctgctttggcagctccagttaatacaaccactgaagatgagactgctcaaatcccagccgaagcagttattggttactccgacttggaaggagattttgacgtcgctgttttaccattctctaattccactaataacggtctgttgtttattaatactaccattgcttctatcgccgctaaggaggaaggtgtgtccctcgagaaaaga

SEQ ID NO: 2 - protein, 85 aa, Artificial Sequence
  PEPTIDE signal peptide
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 3 - DNA, 162 bp, Homo sapiens
  GENE (1)..(162): nucleic acid molecule encoding human insulin analogue precursor
ttcgtcaaccagcacttgtgtggttcccatttggttgaggctctgtacttggtctgtggagaaagaggtttcttttacaccccaaaggctgctaagggtatcgttgagcaatgttgcacctctatttgttccctgtatcagttggaaaactactgcaactaa

SEQ ID NO: 4 - protein, 53 aa, Homo sapiens
  PEPTIDE (1)..(53): human insulin analogue precursor
FVNQHLCGSHLVEALYLVCGERGFFYTPKAAKGIVEQCCTSICSLYQLENYCN

SEQ ID NO: 5 - DNA, 18 bp, Artificial Sequence
  GENE nucleic acid molecule encoding spacer peptide
gaagaaggtgaaccaaag

SEQ ID NO: 6 - DNA, 180 bp, Homo sapiens
gaagaaggtgaaccaaagttcgtcaaccagcacttgtgtggttcccatttggttgaggctctgtacttggtctgtggagaaagaggtttcttttacaccccaaaggctgctaagggtatcgttgagcaatgttgcacctctatttgttccctgtatcagttggaaaactactgcaactaa

SEQ ID NO: 7 - protein, 59 aa, Homo sapiens
EEGEPKFVNQHLCGSHLVEALYLVCGERGFFYTPKAAKGIVEQCCTSICSLYQLENYCN

SEQ ID NO: 8 - DNA, 435 bp, Homo sapiens
atgagatttccttctattttcactgctgttttgtttgccgcttcctctgctttggcagctccagttaatacaaccactgaagatgagactgctcaaatcccagccgaagcagttattggttactccgacttggaaggagattttgacgtcgctgttttaccattctctaattccactaataacggtctgttgtttattaatactaccattgcttctatcgccgctaaggaggaaggtgtgtccctcgagaaaagagaagaaggtgaaccaaagttcgtcaaccagcacttgtgtggttcccatttggttgaggctctgtacttggtctgtggagaaagaggtttcttttacaccccaaaggctgctaagggtatcgttgagcaatgttgcacctctatttgttccctgtatcagttggaaaactactgcaactaa

SEQ ID NO: 9 - protein, 144 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKREEGEPKFVNQHLCGSHLVEALYLVCGERGFFYTPKAAKGIVEQCCTSICSLYQLENYCN

SEQ ID NO: 10 - DNA, 180 bp, Artificial Sequence
  nucleic acid molecule encoding spacer peptide
gaagaaggtgaaccaaagttcgttaaccaacacttgtgcggttcccacttggttgaagctttgtacttggtttgcggtgaaagaggtttcttctacactcctaaggctgctaagggtattgtcgaacaatgctgtacctccatctgctccttgtaccaattggaaaactactgcaactaa

SEQ ID NO: 11 - DNA, 180 bp, Artificial Sequence
  nucleic acid encoding spacer peptide
gaagaaggtgaaccaaagtttgttaaccaacatttgtgtggttctcatttggttgaagctttgtacttggtttgtggtgaaagaggtttcttctacactccaaaggctgctaagggtattgttgaacaatgttgtacttctatttgttctttgtaccaattggaaaactactgtaactaa

SEQ ID NO: 12 - DNA, 255 bp, Artificial Sequence
  GENE nucleic acid molecule encoding a-factor
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaga

SEQ ID NO: 13 - DNA, 435 bp, Artificial Sequence
  GENE nucleic acid encoding a-factor + nucleic acid encoding insulin analogue precursor
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaagaaggtgaaccaaagttcgtcaaccagcacttgtgtggttcccatttggttgaggctctgtacttggtctgtggagaaagaggtttcttttacaccccaaaggctgctaagggtatcgttgagcaatgttgcacctctatttgttccctgtatcagttggaaaactactgcaactaa

SEQ ID NO: 14 - DNA, 435 bp, Artificial Sequence
  GENE nucleic acid encoding a-factor + nucleic acid encoding insulin analogue precursor
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaagaaggtgaaccaaagttcgttaaccaacacttgtgcggttcccacttggttgaagctttgtacttggtttgcggtgaaagaggtttcttctacactcctaaggctgctaagggtattgtcgaacaatgctgtacctccatctgctccttgtaccaattggaaaactactgcaactaa

SEQ ID NO: 15 - DNA, 435 bp, Artificial Sequence
  GENE nucleic acid encoding a-factor + nucleic acid encoding insulin analogue precursor
atgagatttccttctattttcactgctgttttgtttgccgcttcctctgctttggcagctccagttaatacaaccactgaagatgagactgctcaaatcccagccgaagcagttattggttactccgacttggaaggagattttgacgtcgctgttttaccattctctaattccactaataacggtctgttgtttattaatactaccattgcttctatcgccgctaaggaggaaggtgtgtccctcgagaaaagagaagaaggtgaaccaaagttcgttaaccaacacttgtgcggttcccacttggttgaagctttgtacttggtttgcggtgaaagaggtttcttctacactcctaaggctgctaagggtattgtcgaacaatgctgtacctccatctgctccttgtaccaattggaaaactactgcaactaa

SEQ ID NO: 16 - protein, 6 aa, Artificial Sequence
  PEPTIDE spacer peptide
EEGEPK